(19) United States
(12) Patent Application Publication
(10) Pub. No.: US 2026/0062390 A1
(43) Pub. Date: Mar. 5, 2026

(54) CONTINUOUS FLOW SYNTHESIS OF LORAZEPAM

(71) Applicants: Purdue Research Foundation, West Lafayette, IN (US); Continuity Pharma, LLC, West Lafayette, IN (US)

(72) Inventors: David Thompson, West Lafayette, IN (US); Shruti Biyani, Midland, MI (US); Michael McGuire, Lafayette, IN (US); Seok-Hee Hyun, West Lafayette, IN (US)

(21) Appl. No.: 19/102,315

(22) PCT Filed: Aug. 7, 2023
(86) PCT No.: PCT/US2023/071797

§ 371 Date: Feb. 7, 2025

Related U.S. Application Data
(60) Provisional application No. 63/396,034, filed on Aug. 8, 2022.

(51) Int. Cl. C07D 243/28

(52) U.S. Cl. CPC C07D 243/28

(57) ABSTRACT

A method for synthesis of Lorazepam in a continuous flow using continuous flow reactor, in which method comprises five steps including N-acylation, diazepine ring closure, imine N-oxidation, Polonovski-type rearrangement, and ester hydrolysis; a green reagent comprising a peroxide reagent and rhenium oxide catalyst for N-oxidation of delorazepam; and an ammonium source combination for the synthesis of delorazepam.

### CROSS-REFERENCE TO RELATED APPLICATION

This application claims priority to U.S. provisional patent application No. 63/396,034, which was filed Aug. 8, 2022, and which is hereby incorporated by reference in its entirety.

### GOVERNMENT RIGHTS

This invention was made with government support under HR0011-20-C-0199 awarded by the Defense Advanced Research Projects Agency. The government has certain rights in the invention.

### TECHNICAL FIELD

The present disclosure relates to a continuous flow microfluidic synthesis of benzodiazepines such as Lorazepam. This green synthesis reduces the overall production time and avoids batch production by using a continuous flow reactor system.

### BACKGROUND

Drug shortage is a global threat, with more than a hundred medicines experiencing shortages in the United States alone in 2020. Lorazepam, also known as (7-chloro-5-(2-chlorophenyl)-3-hydroxy-1,3-dihydro-2H-benzo[e][1,4]diazepin-2-one), is one of the most used benzodiazepines for sedation. It is classified as one of the essential medicines by the World Health Organization, which experiences periodic shortages. Lorazepam is commonly used as a sedative, anti-anxiety agent, and hypnotic agent in the treatment of epilepsy.

Lorazepam shortages have affected hospitals, dental practices, and critically ill patients that require sedation and intubation for their life-saving care. The reasons listed for the short supply of Lorazepam include manufacturing delays, a limited number of manufacturers, manufacturer supply shortages, increased demand for the drug, and discontinuation of drug production sites. The coronavirus pandemic has aggravated the Lorazepam shortage problem due to significant increases in the number of patients requiring intubation, thereby increasing the demand for this sedative.

The reported synthesis of Lorazepam in batch suffers from process irreproducibility issues, inability to scale readily, and the use of harsh oxidizing agents like persulfate. Some of the syntheses require additional steps that are time consuming. Batch processes can be challenging to scale up, as they require a large footprint, long hold times, transportation, and reactor scheduling. There are also safety challenges to increase productivity to meet the increased demand for an active pharmaceutical ingredient (API) in shortage.

Therefore, there is an unmet need for a robust and efficient process for the synthesis of Lorazepam that offers the potential for an accelerated scale-up from proof-of-concept to large-scale manufacturing of the API consistently with high quality and time reduction by executing it in flow. It is an object of the present disclosure to provide such a process. This and other objects and advantages, as well as inventive features, will be apparent from the detailed description.

### SUMMARY

A method for a continuous flow synthesis of lorazepam of formula (1), also chemically known as (7-chloro-5-(2-chlorophenyl)-3-hydroxy-1,3-dihydro-2H-benzo[e][1,4]diazepin-2-one), is provided.

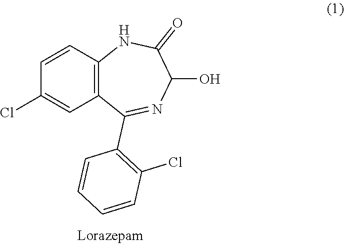 [SMILES: O=C1Nc2ccc(Cl)cc2C(c2ccccc2Cl)=NC1O]

In some embodiments, the method for a continuous flow synthesis of lorazepam (1) is carried out in a continuous flow reactor and comprises:
(a) acylating 2-amino-2′,5-dichlorobenzophenone (2) by mixing a solution comprising (2)

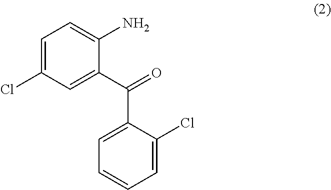 [SMILES: Nc1ccc(Cl)cc1C(=O)c1ccccc1Cl]

with a solution comprising an acylating agent and a solution comprising an acid scavenger to yield 2-halo-N-(4-chloro-2-(2-chlorobenzoyl)phenyl)acetamide (halo intermediate, (3));

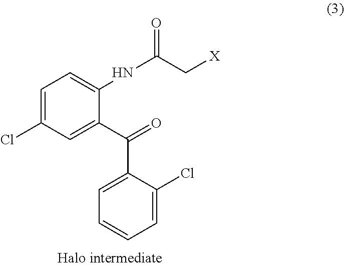

wherein X is a halogen comprising Cl or Br;
(b) cyclizing the halo intermediate (3) by mixing a solution comprising the halo intermediate (3) with a solution comprising an ammonium reagent to yield 7-chloro-5-(2-chlorophenyl)-1,3-dihydro-2H-benzo[e][1,4]diazepin-2-one (delorazepam, (4));

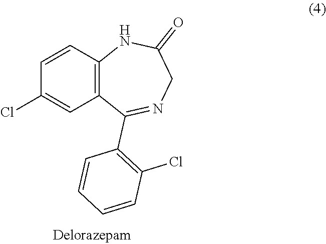 [SMILES: O=C1CN=C(c2ccccc2Cl)c2cc(Cl)ccc2N1]

(c) oxidizing delorazepam (4) by mixing a solution comprising delorazepam (4) with a solution comprising a peroxide reagent and a solution comprising a rhenium oxide catalyst to yield 7-chloro-5-(2-chlorophenyl)-2-oxo-2,3-dihydro-1H-benzo[e][1,4]diazepine 4-oxide (delorazepam N-oxide, (5));

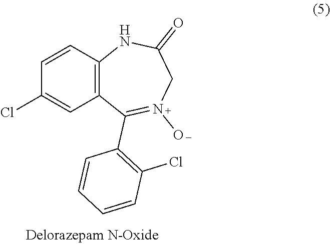

(d) mixing a solution comprising delorazepam N-oxide (5) with a solution comprising an acylating reagent for performing Polonovski-type rearrangement to yield 7-chloro-5-(2-chlorophenyl)-2-oxo-2,3-dihydro-1H-benzo[e][1,4]diazepin-3-yl acetate (lorazepam acetate, (6));

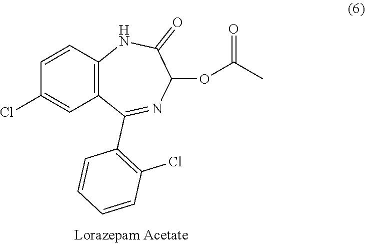 [SMILES: CC(=O)OC1N=C(c2ccccc2Cl)c2cc(Cl)ccc2NC1=O]

and
(e) hydrolyzing lorazepam acetate (6) by mixing a solution comprising lorazepam acetate (6) and a solution comprising a base and an additive to yield lorazepam (1).

Any suitable continuous flow reactor as is known in the art can be used for the continuous flow synthesis of lorazepam (1). Examples of suitable continuous flow reactors include, but are not limited to, a plug flow reactor, a segmented flow reactor, and a continuous stirred tank reactor. This reactor can be a glass reactor, a ceramic reactor, a coiled flow inverter reactor, a coiled tubing reactor, a packed bed reactor, an oscillatory, a spinning disc, or a 3D-printed type reactor, for example.

The continuous flow reactor comprises a mixer, examples of which include a T-mixer, a Y-mixer, a static mixer, an ultrasonic mixer, a staggered oriented ridge mixer, a zig-zag mixer, a packed bed mixer, and a coiled flow inverter mixer.

An acylation of 2-amino-2′,5-dichlorobenzophenone (2) in step (a), with an acylating agent in the presence of an acid scavenger to obtain a halo intermediate (3), is provided. The acylating agent can be any suitable acylating agent as is known in the art. Examples of suitable acylating agents include, but are not limited to, bromoacetyl bromide, bromoacetyl chloride, 2-bromoacetic acid, bromoacetic anhydride, chloroacetyl chloride, and ethyl bromoacetate. The acid scavenger can be any suitable acid scavenger as known in the art, examples of which include propylene oxide, ethylene oxide, and butylene oxide. Examples of solvents include, but are not limited to, acetonitrile, 2-methyltetrahydrofuran(2-MeTHF), ethyl acetate, toluene, N-methyl pyrrolidone, water, dichloromethane, acetone, acetic acid, and combinations thereof. For this step, the residence time range can be from about 30 sec. to about 30 min. (such as from about 30 seconds to 30 minutes or from 30 seconds to about 30 minutes), and the temperature range can be from about −20° C. to about 100° C. (such as from about −20° C. to 100° C. or −20° C. to about 100° C.).

Provided is a cyclization of halo intermediate (3) in step (b), using an ammonium reagent to obtain delorazepam (4). In exemplary embodiments, the ammonium reagent can be ammonium chloride, ammonium iodide, ammonium bromide, ammonium hydroxide, ammonium acetate, or a combination thereof. In exemplary embodiments, the ammonium reagent can be a mixture of ammonium bromide and ammonium hydroxide, or a mixture of ammonium iodide, ammonium acetate, and ammonium chloride. The residence time range can be from about 30 sec. to about 60 min. (such as from about 30 seconds to 60 minutes or from 30 seconds to about 60 minutes), and the temperature range can be from about 20° C. to about 180° C. (such as from about 20° C. to 180° C. or 20° C. to about 180° C.). Examples of solvents include, but are not limited to, acetonitrile, N-methyl pyrrolidone, methanol, ethyl acetate, acetone, ethanol, toluene, dicholormethane, butanone, water, dimethyl sulfoxide, acetic acid, N-methyl pyrrolidone 2-MeTHF, and combinations thereof.

N-oxidation of delorazepam (4) in step (c), using a peroxide reagent and a rhenium oxide catalyst to obtain delorazepam N-oxide (5), is provided. In exemplary embodiments, the peroxide reagent can be urea-hydrogen peroxide, magnesium monoperoxyphthalate, hydrogen peroxide (H₂O₂), peroxyacetic acid, tert-butyl hydroperoxide, or sodium percarbonate. In exemplary embodiments, the rhenium oxide catalyst can be methyl rhenium trioxide (CH₃ReO₃) or rhenium oxide (Re₂O₇). The residence time range can be from about 5 min. to about 12 hours (such as from about 5 minutes to 12 hours or from 5 minutes to about 12 hours), and the temperature range can be from about 20° C. to about 150° C. (such as from about 20° C. to 150° C. or from 20° C. to about 150° C.). Examples of solvents include, but are not limited to, anhydrous methanol, methanol, 2-MeTHF, ethyl acetate, ethanol, propanol, iso-propanol, butanol, acetone, acetic acid, and combinations thereof.

Provided is an acylation of delorazepam N-oxide (5) in step (d), using an acylating reagent by Polonovski-type rearrangement to yield lorazepam acetate (6). The acylating reagent can be any suitable acylating agent as is known in the art. The acylating reagent can be acetic anhydride, trifluoroacetic anhydride, or acetyl chloride, for example. The residence time range can be from about 1 min. to about 30 min. (such as from about 1 minute to 30 minutes or 1 minute to about 30 minutes), and the temperature range can be from about 50° C. to about 150° C. (such as from about 50° C. to 150° C. or from 50° C. to about 150° C.). Examples of solvents include, but are not limited to, acetic acid, ethyl acetate, acetonitrile, toluene, dichloromethane, water, methanol, ethanol, toluene, dichloromethane, and combinations thereof.

Hydrolysis of lorazepam acetate (6) in step (e), using a base in the presence of an additive to yield lorazepam (1), is provided. Examples of the base that can be used include, but are not limited to, ammonium hydroxide, sodium hydroxide, aluminum hydroxide, magnesium hydroxide, potassium hydroxide, ammonia, 7 M ammonia in methanol (MeOH), triazabicyclodecene, triethylamine, and ammonium acetate. Examples of the additives that can be used include, but are not limited to, ammonium acetate, sodium acetate, potassium acetate, aluminum acetate dibasic, and acetate buffer. The residence time range can be from about 1 min. to about 120 min. (such as from about 1 minute to 120 minutes or from 1 minute to about 120 minutes), and the temperature range can be from about −20° C. to about 100° C. (such as from about −20° C. to 100° C. or from −20° C. to about 100° C.). Examples of solvents include, but are not limited to, N,N′-dimethyl formamide (DMF), N,N′-dimethyl acetamide, ethanol, methanol, butanol, iso-propanol, water, acetonitrile N-methyl pyrrolidone, and combinations thereof.

Provided is a new reagent for N-oxidation of delorazepam (4) for synthesis of lorazepam (1), wherein the new reagent is a combination of a peroxide reagent and a rhenium oxide catalyst. In exemplary embodiments, the peroxide reagent that can be used includes, but is not limited to, urea-hydrogen peroxide, magnesium monoperoxyphthalate, H₂O₂, peroxyacetic acid, tert-butyl hydroperoxide, and sodium percarbonate. In exemplary embodiments, the rhenium oxide catalyst can be CH₃ReO₃, or Re₂O₇.

An ammonium reagent for cyclizing halo intermediate (3) for synthesis of lorazepam (1) is provided. The ammonium reagent can comprise ammonium chloride, ammonium iodide, ammonium bromide, ammonium hydroxide, ammonium acetate, or a combination thereof. In exemplary embodiments, the ammonium reagent can be a mixture of ammonium bromide and ammonium hydroxide or a mixture of ammonium iodide, ammonium acetate, and ammonium chloride.

Provided is the method for synthesis of lorazepam (1) in continuous flow using the continuous flow reactor, where the mean residence time for each of the individual flow reactions can add up to a total of about 72.5 min. (such as 72.5 minutes). The method provides the final product lorazepam (1) with higher yield and purity by using flow reactions.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

The term “halo,” is used to include chemical compounds which contain one or more halogen atoms, such as fluorine, chlorine, bromine, and iodine.

The term “residence time” is the time for which the reaction solution resides in the flow reactor. It is calculated by using the formula volume of the reactor/total volumetric flow rate through the reactor.

Continuous flow synthesis offers an accelerated scale-up to large-scale manufacturing of an active pharmaceutical ingredient consistently with high quality. Continuous flow reactors provide efficient heat and mass transfer due to their high surface area-to-volume ratios as well as precise control over reaction parameters like temperature, stoichiometry, and residence time with increased safety and improved yield. They also offer the potential for the development of improved synthesis routes, resulting in reduced E-factors relative to batch processes. Continuous flow synthesis provides a significantly shorter time than the overall time required for batch synthesis. These steps can be either telescoped or performed individually.

Lorazepam, chemically known as (7-chloro-5-(2-chlorophenyl)-3-hydroxy-1,3-dihydro-2H-benzo[e][1,4]diazepin-2-one), is represented by formula (1):

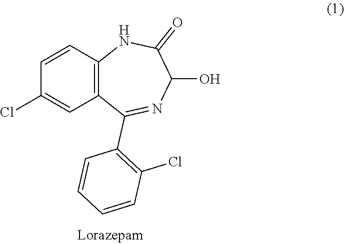 [SMILES: O=C1Nc2ccc(Cl)cc2C(c2ccccc2Cl)=NC1O]

In some embodiments, provided is a method for synthesis of lorazepam (1) in a continuous flow reactor, which method comprises:
(a) acylating 2-amino-2′,5-dichlorobenzophenone (2) by mixing a solution comprising (2)

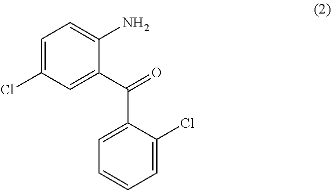 [SMILES: Nc1ccc(Cl)cc1C(=O)c1ccccc1Cl]

with a solution comprising an acylating agent, and a solution comprising an acid scavenger to yield 2-halo-N-(4-chloro-2-(2-chlorobenzoyl)phenyl)acetamide (halo intermediate, (3));

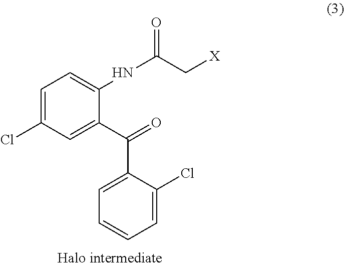

wherein X is a halogen comprising Cl or Br;
(b) cyclizing the halo intermediate (3) by mixing a solution comprising the halo intermediate (3) with a solution comprising an ammonium reagent to yield 7-chloro-5-(2-chlorophenyl)-1,3-dihydro-2H-benzo[e][1,4]diazepin-2-one (delorazepam, (4));

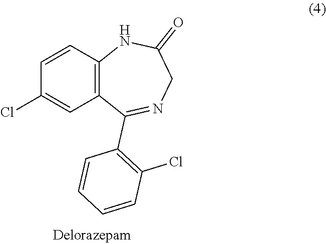 [SMILES: O=C1CN=C(c2ccccc2Cl)c2cc(Cl)ccc2N1]

(c) oxidizing delorazepam (4) by mixing a solution comprising delorazepam (4) with a solution comprising a peroxide reagent and a solution comprising a rhenium oxide catalyst to yield 7-chloro-5-(2-chlorophenyl)-2-oxo-2,3-dihydro-1H-benzo[e][1,4]diazepine 4-oxide (delorazepam N-oxide, (5));

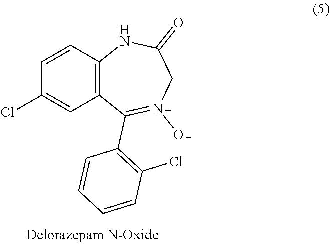

(d) mixing a solution comprising delorazepam N-oxide (5) with a solution comprising an acylating reagent for performing Polonovski-type rearrangement to yield 7-chloro-5-(2-chlorophenyl)-2-oxo-2,3-dihydro-1H-benzo[e][1,4]diazepin-3-yl acetate (lorazepam acetate, (6));

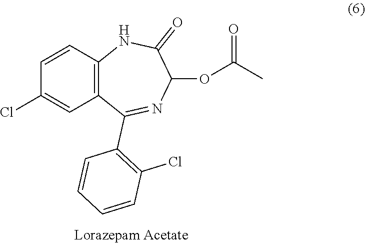 [SMILES: CC(=O)OC1N=C(c2ccccc2Cl)c2cc(Cl)ccc2NC1=O]

and
(e) hydrolyzing lorazepam acetate (6) by mixing a solution comprising lorazepam acetate
(6) and a solution comprising a base and an additive to yield lorazepam (1).

This method of continuous flow synthesis of lorazepam involves green chemistry principles, including improved safety, solvent bio-renewability and biodegradability, and compound solubility. None of the steps requires column chromatography for compound isolation. It provides lorazepam in higher yield and purity by using flow reactions that are optimized using high-throughput experimentation and rapid flow chemistry. The method for continuous flow synthesis of lorazepam can be completed with a mean residence time for each of the individual flow reactions added up to a total of about 72.5 min. (such as 72.5 minutes).

The residence time for each step is calculated based on a volume of a reactor and volumetric flow rate of reactants. The flow rate for each reaction is set using a Chemtrix software, which is used in the continuous flow reactor.

Provided is a new ammonium reagent for a ring closure cyclization of a halo intermediate (3) in the synthesis of lorazepam (1). The ammonium reagent can be a mixture of ammonia source comprising ammonium chloride, ammonium iodide, ammonium bromide, ammonium hydroxide, ammonium acetate or a combination thereof. In exemplary embodiments, the ammonia reagent can be a mixture of ammonium bromide and ammonium hydroxide, or a mixture of ammonium iodide, ammonium acetate, and ammonium chloride.

Provided is a new reagent for N-oxidation of delorazepam (4) in the synthesis of lorazepam (1), wherein the reagent comprises combination of a peroxide reagent and a rhenium oxide catalyst. In exemplary embodiments, the peroxide reagent that can be used includes, but is not limited to, urea-hydrogen peroxide, magnesium monoperoxyphthalate, hydrogen peroxide (H₂O₂), peroxyacetic acid, tert-butyl hydroperoxide, and sodium percarbonate. In exemplary embodiments, the rhenium oxide catalysts can be methyl rhenium trioxide (CH₃ReO₃), or rhenium oxide (Re₂O₇). Example of the suitable reagent can be the combination of CH₃ReO₃ with urea-hydrogen peroxide (urea-H₂O₂).

Urea-H₂O₂ is a safer and greener peroxide that is easier to handle in the presence of CH₃ReO₃ catalyst. It provides efficient oxidation with about 97% purity of the desired N-oxide of delorazepam (4) without any need for chromatographic purification.

Scheme 1 shows the method for synthesis of lorazepam (1) in the continuous flow reactor. The method comprises five steps, starting with a benzophenone precursor, 2-amino-2′,5-dichlorobenzophenone(2).

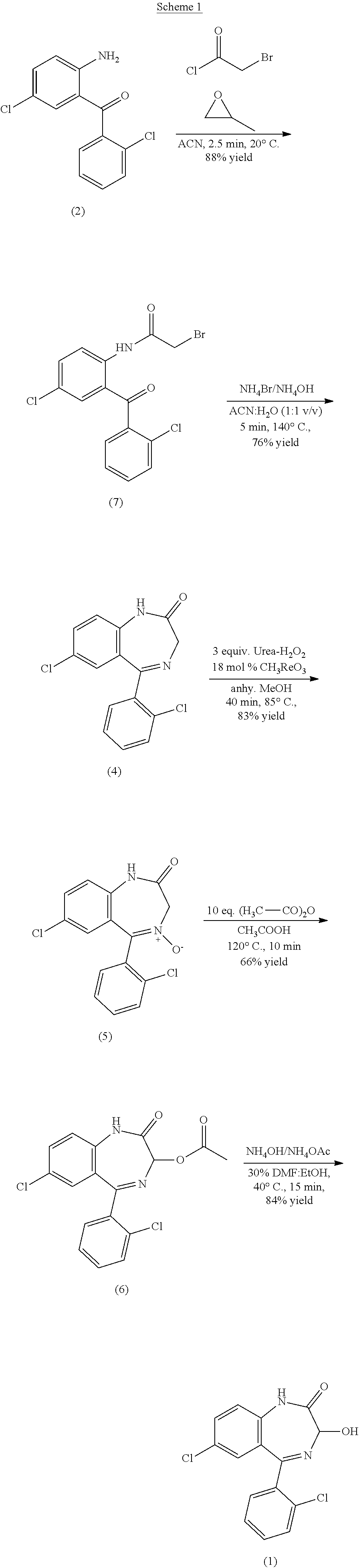

Scheme 2 illustrates the step (a) for N-acylation of 2-amino-2′,5-dichlorobenzophenone (2). The acylation was carried out by mixing the solution comprising 2, the solution comprising the acylating agent, and the solution comprising the acid scavenger to obtain a bromo intermediate, 2-bromo-N-(4-chloro-2-(2-chlorobenzoyl)phenyl)acetamide (bromo intermediate, (7)) in the continuous flow reactor.

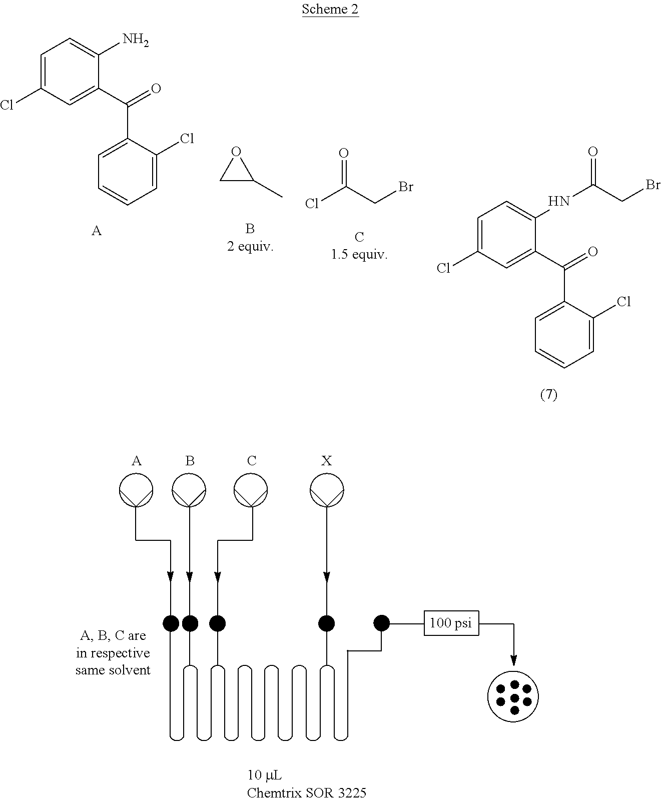

The flow synthesis can be carried out using a Chemtrix 3225 glass chip reactor (10 μL staggered oriented ridge). Full-factorial design (2²) was performed in the flow reactions to identify quickly the optimal choice of solvent in the flow synthesis for the N-acylation reaction. Different solvents such as N-methyl pyrrolidone (NMP), 2-methyl tetrahydrofuran (2-MeTHF), ethyl acetate (EtOAc), and toluene were tried, alone or in combinations, based on the solubility of the halo intermediate. Also, the temperature was varied from 0° C. to 80° C. with residence times of 1, 5, and 10 min.

The reaction was carried out using another solvent acetonitrile (ACN). The efficient reaction condition with ACN solvent was at residence time of about 2.5 minutes with a temperature at about 20° C. to yield 95% of halo intermediate (3) by ultra-pressure liquid chromatography (UPLC) analysis. The results of the optimization of the reaction in ACN are summarized in Table 1. 2-MeTHF also yields an efficient reaction at a temperature of about 20° C. and at a residence time of about 10 minutes.

TABLE 1

| | R_{T} | Temp | UPLC Area % | UPLC Area % | UPLC Area % |
|---|---|---|---|---|---|
| Entry | (min) | (° C.) | (7) | (8) | (2) |
| 1 | 1^{a} | 0 | 35 | 1 | 64 |
| 2 | 10^{a} | 0 | 46 | 1 | 50 |
| 3 | 2.5 | 20 | 95 | 5 | 0 |
| 4 | 5 | 20 | 95 | 5 | 0 |

0.135M Benzophenone 1, 0.27M propylene oxide (2 equiv.), 0.2025M bromoacetyl chloride (1.5 equiv.). All compounds were dissolved in HPLC grade ACN. Each collection vial contained 850 μL ACN prior to sample collection. The product was collected and immediately frozen at −80° C.
^{a}1.25 equiv. bromoacetyl chloride.
(2) is benzophenone, (7) is bromo intermediate, and (8) is chloro intermediate

Examples of the solvents that can be used in N-acylation in step (a) include, but are not limited to, acetonitrile, 2-MeTHF, ethyl acetate, toluene, N-methyl pyrrolidone, water, dichloromethane, acetone, and a combination thereof. Examples of the combination of solvents can include, but are not limited to, acetonitrile and N-methyl pyrrolidone, or acetonitrile, acetic acid, and 2-MeTHF. In exemplary embodiments, the most suitable solvent can be ACN.

The acylating reagent can be any suitable acylating reagent as is known in the art. Examples of the suitable acylating reagents for N-acylation in step (a) can include, but are not limited to, bromoacetyl bromide, bromoacetyl chloride, 2-bromoacetic acid, bromoacetic anhydride, chloroacetyl chloride, and ethyl bromoacetate.

The acid scavengers can be any suitable acid scavengers as is known in the art. Examples of the suitable acid scavengers that can be used in N-acylation in step (a) include, but are not limited to, propylene oxide, ethylene oxide, and butylene oxide.

The residence time for N-acylation in step (a) can range from about 30 sec. to about 30 min., such as about 30 sec. to 30 min. or 30 sec. to about 30 min. In exemplary embodiments, the suitable residence time can range from about 1 min. to about 10 min., such as from about 1 min. to 10 min. or 1 min. to about 10 min.

The temperatures for N-acylation in step (a) can range from about −20° C. to about 100° C., such as from about −20° C. to 100° C. or −20° C. to about 100° C. In exemplary embodiments, the suitable temperature can range from about 0° C. to about 100° C., preferably from about 0° C. to about 40° C.

Stoichiometry of the acylating agents for N-acylation in step (a) can range from about 1 equivalent to about 5 equivalents, such as about 1 equivalent to 5 equivalents or 1 equivalent to about 5 equivalents.

Stoichiometry of the acid scavenger can range from about 0.5 equivalent to about 5 equivalents, such as from about 0.5 equivalent to 5 equivalents or 0.5 equivalent to about 5 equivalents.

The cyclization in step (b) to form the 1,4-diazepine ring is reported with hexamethylene tetramine in a two-step process (Hannoun, M. et. al. Synthesis of imidazolidin-4-ones and their conversion into 1,4-benzodiazepin-2-ones. J. Het. Chem. 1981, 18, 963-965). High throughput experimentation with different ammonium sources in ethanol such as ammonium hydroxide, ammonium iodide, ammonium bromide, ammonium chloride, ammonium acetate, ammonium carbonate, ammonium sulfate, ammonium citrate dibasic, or 7 M ammonia in methanol (MeOH) in presence and absence of hexamine were studied. The experiment was performed in the 96-well heating block using the Biomek i7 liquid handling robot. The reaction mixture was analyzed using Desorption Electrospray Ionization-Mass Spectrometry (DESI-MS).

Scheme 3 illustrates step (b) for the cyclization of the bromo intermediate (7) using the ammonia reagent to yield delorazepam (4), wherein the 1,4-diazepine ring is formed in a one-step process in the continuous flow reactor.

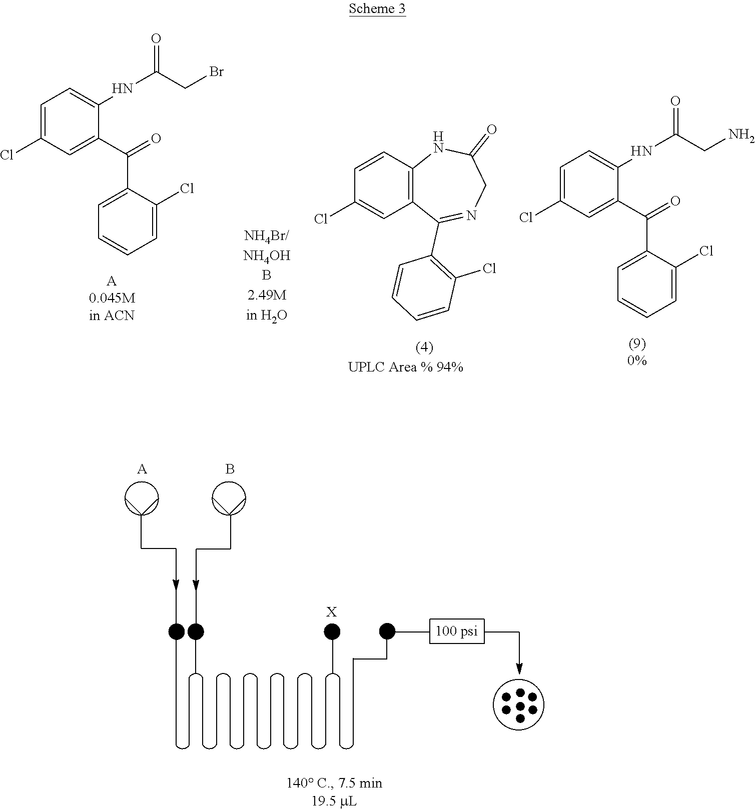

The cyclization can be carried out using a Chemtrix glass reactor chip 3227 (19.5 μL with a staggered oriented ridge mixer). The various ammonium reagents from high throughput experimentation and their combinations were studied. The cyclization reaction was executed in the flow reactor at various residence times and temperatures. The results are summarized in Table 2.

TABLE 2

| | | | UPLC | UPLC | | UPLC |
|---|---|---|---|---|---|---|
| | | | area % | area % | UPLC | area % |
| | | | ring opening | Delor- | area % | bromo |
| En- | R_{T} | Temp | intermediate | azepam | Unknown | intermediate |
| try | (min) | (° C.) | (9) | (4) | impurity | (7) |
| 1 | 5 | 60 | 19 | 0 | 3.4 | 56 |
| 2 | 1 | 100 | 31 | 53 | 0 | 7 |
| 3 | 5 | 100 | 66 | 30 | 0.5 | 3 |
| 4 | 1 | 120 | 69 | 17 | 2.2 | 9.4 |
| 5 | 2.5 | 120 | 44 | 50 | 4.3 | 0 |
| 6 | 5 | 120 | 23 | 72 | 4 | 0 |
| 7 | 1 | 140 | 49 | 39 | 3 | 6.5 |
| 8 | 2.5 | 140 | 13 | 83 | 3.7 | 0 |
| 9 | 5 | 140 | 0 | 94 | 3.8 | 0 |
| 10 | 7.5 | 140 | 0 | 94 | 3.8 | 0 |
| 11 | 1.5 | 160 | 26 | 57 | 15 | 1.5 |
| 12 | 5 | 160 | 36 | 40 | 23 | 1.8 |
| 13 | 1 | 180 | 21 | 26.5 | 20 | 2.7 |

0.045M bromo intermediate (7) in ACN, 1.16 g NH₄Br with 1.4 mL 30% NH₄OH was used in the flow system.

The delorazepam (4) purity, determined by UPLC analysis using 254 nm detection, was 94%. The reaction showed no formation of ring open intermediate (9) with the solvents ACN or mixtures of ACN and water. Product formation is increased from 60° C. to 140° C., with a decrease in the ring-opened intermediate (9) and an increase in the cyclized product (4). The optimal conditions can be achieved at 140° C. to yield 94% delorazepam (4), with a residence time (R_{T}) of 5 min (Table 2, entry 9).

Examples of the solvents that can be used for the cyclization include, but are not limited to, acetonitrile, N-methyl pyrrolidone, methanol, ethyl acetate, acetone, ethanol, toluene, dichloromethane, butanone, water, ethanol, dimethyl sulfoxide (DMSO), acetic acid, and a combination thereof. Examples of a combination of solvents that can be used include, but are not limited to, acetonitrile and water; N-methyl pyrrolidone, acetonitrile, and water; 2-MeTHF, acetonitrile, and water; or ethanol, acetonitrile, and water.

The ammonia reagents that can be used for cyclization include, but are not limited to, ammonium bromide, ammonium hydroxide, ammonium iodide, ammonium acetate, ammonium chloride, and a combination thereof. In exemplary embodiments, the ammonium reagent can be a mixture of ammonium bromide and ammonium hydroxide, or a mixture of ammonium iodide, ammonium acetate, and ammonium chloride. In some embodiments, the preferred ammonium reagent can be a mixture of ammonium bromide and ammonium hydroxide.

The residence time for cyclization can range from about 30 sec. to about 60 min., such as from about 30 sec. to 60 min. or 30 sec. to about 60 min. In exemplary embodiments, the suitable residence times can range from about 1 min. to about 10 min., preferably about 5 min.

The reaction temperature for the cyclization can range from about 20° C. to about 180° C., such as from about 20° C. to 180° C. or 20° C. to about 180° C. In exemplary embodiments, the suitable temperature can range from about 40° C. to about 180° C., preferably about 60° C. to about 140° C.

High throughput experimentation was performed for the selection of the reagent for the synthesis of an intermediate delorazepam N-oxide by oxidation of delorazepam. The various peroxide reagents, such as cumene hydroperoxide, H₂O₂, manganese monoperoxyphthalate, cumene hydroperoxide, and urea-H₂O₂, were tested for the N-oxidation step.

Delorazepam (4) is oxidized in step (c) by mixing the solution comprising delorazepam with the solution comprising the peroxide reagent and rhenium oxide as a catalyst in the continuous flow reactor, as shown in the Scheme 4 below. Step (c) was carried out using a Chemtrix glass reactor chip 3227 with or without a T-mixer. The constant gas formation was observed when the oxidation step was carried out without the T-mixer. Use of the T-mixer eliminated the formation of gas and delivered a consistent flow. The reaction was optimized with different residence times of 10, 20, 40, and 60 min. and solvents at 85° C. No N-oxide product was obtained with MeOH at R_{T}=10 min. Anhydrous MeOH yielded 36% N-oxide product at R_{T}=10 min. Swapping anhydrous MeOH to HPLC grade MeOH gave similar results. The presence of adventitious water can result in the degradation of the catalyst.

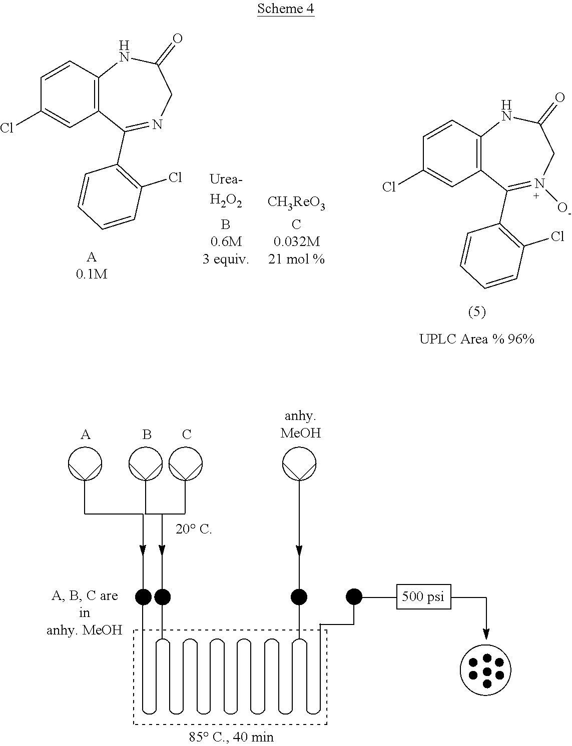

The optimal conditions were achieved by increasing the amount of CH₃ReO₃ catalyst to 21% at a residence time of 40 min. to yield Delorazepam N-oxide (5) in 97% purity.

The peroxide reagents that can be used in the N-oxidation include, but are not limited to, urea-hydrogen peroxide, magnesium monoperoxyphthalate, hydrogen peroxide, peroxyacetic acid, tert-butyl hydroperoxide and sodium percarbonate. In some embodiments, the preferred peroxide reagent can be urea-hydrogen peroxide.

The rhenium oxide catalysts used in the N-oxidation can include, but are not limited to, CH₃ReO₃, and Re₂O₇. In some embodiments, the preferred catalyst can be methyl rhenium trioxide.

The amount of catalyst used in the N-oxidation can range from about 0.1 mol % to about 2 equivalents, such as from about 0.1 mol % to 2 equivalents or 0.1 mol % to about 2 equivalents.

Examples of the solvents used in the N-oxidation can include, but are not limited to, anhydrous methanol, methanol, 2-MeTHF, ethyl acetate, ethanol, propanol, iso-propanol, butanol, acetone, or a combination thereof. Examples of a combination of solvents can include, but are not limited to, methanol and ethyl acetate, or ethyl acetate and acetic acid.

The residence time for the N-oxidation can range from about 5 min. to about 3 hours, such as from about 5 minutes to 3 hours or 5 minutes to about 3 hours.

The reaction temperature for the N-oxidation can range from about 20° C. to about 150° C., such as from about 20° C. to 150° C. or 20° C. to about 150° C.

Scheme 5 illustrates the Polonovski-type rearrangement of delorazepam N-oxide (5) by mixing the solution comprising delorazepam N-oxide with the solution comprising the acylating agent to yield lorazepam acetate (6) in the continuous flow reactor. The step (d) can be carried out using a Chemtrix glass reactor chip 3227 (19.5 μL with a staggered oriented ridge mixer).

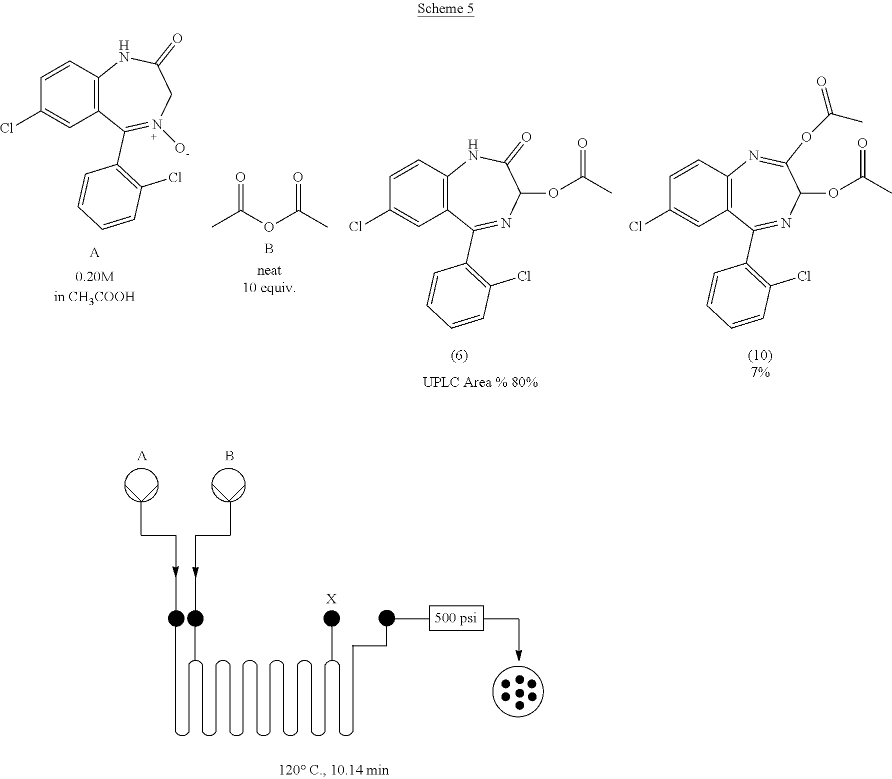

This reaction was tried in various solvents. The solvent 2-MeTHF led to multiple byproducts, while over 95% N-oxide remained when acetone was used as the solvent. The reactivity depended on different experimental parameters such as residence time, temperature, and stoichiometry. A 2³ full-factorial design approach was used to study the reactivity with parameters such as residence times of 1 min. and 5 min. and temperatures of 60° C. and 120° C., with acetic acid equivalents of 3 equiv. and 10 equiv. The results are summarized in Table 3.

TABLE 3

| | | | Equiv. | UPLC | UPLC | UPLC |
|---|---|---|---|---|---|---|
| | | | of | Area % | Area % | Area % |
| | | | Acetic | Lorazepam | Lorazepam | Delorazepam |
| En- | R_{T} | Temp | Anhy- | Acetate | Diacetate | N-oxide |
| try | (min) | (° C.) | dride | (6) | (10) | (5) |
| 1 | 1 | 60 | 3 | 0.3 | 0 | 90 |
| 2 | 1 | 60 | 10 | 5 | 0 | 87 |
| 3 | 1 | 120 | 3 | 38 | 0.3 | 48 |
| 4 | 1 | 120 | 10 | 41 | 0.6 | 52 |
| 5 | 5 | 60 | 3 | 13 | 1.4 | 71 |
| 6 | 5 | 60 | 10 | 12 | 0 | 83 |
| 7 | 5 | 120 | 3 | 38 | 0.3 | 50 |
| 8 | 5 | 120 | 10 | 77 | 3 | 13 |
| 9 | 10.14 | 120 | 10 | 80 | 7 | 6 |

Delorazepam N-oxide (5) was largely unconsumed at 60° C. (Table 3, Entry 1-2 and 5-6), whereas maximum product formation was obtained at 120° C., R_{T}=5 min, and 10 equiv. of acetic anhydride. Another reaction was carried out at 120° C. using R_{T}=10 min. gave an overall 87% lorazepam acetate (6) and diacetate (10) yield. Both the intermediates 6 and 10 lead to lorazepam (1) upon hydrolysis with ammonium hydroxide as a base.

The acylating reagent used for the Polonovski-type rearrangement can be any suitable acylating agent as is known in the art. The acylating reagents can include, but are not limited to, acetic anhydride, trifluoroacetic anhydride, trifluoro methane sulfonic anhydride, and acetyl chloride.

The amount of acylating reagent used for the Polonovski-type rearrangement can range from about 0.5 mol % to about 30 equivalents, such as about 0.5 mol % to 30 equivalents or 0.5 mol % to about 30 equivalents.

The examples of the solvents that can be used for the Polonovski-type rearrangement include, but are not limited to, acetic acid, ethyl acetate, acetonitrile, toluene, dichloromethane, and a combination thereof. The examples of a combination of solvents can include, but are not limited to, mixtures of acetic acid with the organic solvents comprising ethyl acetate, water, methanol, ethanol, toluene, and dichloromethane.

The residence times for the Polonovski-type rearrangement can range from about 1 min. to about 30 min., such as from about 1 min. to 30 min. or 1 min. to about 30 min.

The reaction temperature for the Polonovski-type rearrangement can range from about 50° C. to about 150° C., such as from about 50° C. to 150° C. or 50° C. to about 150° C.

Scheme 6 shows the hydrolysis of lorazepam acetate (6) by mixing the solution comprising lorazepam acetate with the solution comprising the base and the additive to yield lorazepam (1) in the continuous flow reactor. The flow reactor can be a Chemtrix glass reactor chip 3227 (19.5 μL staggered oriented ridge).

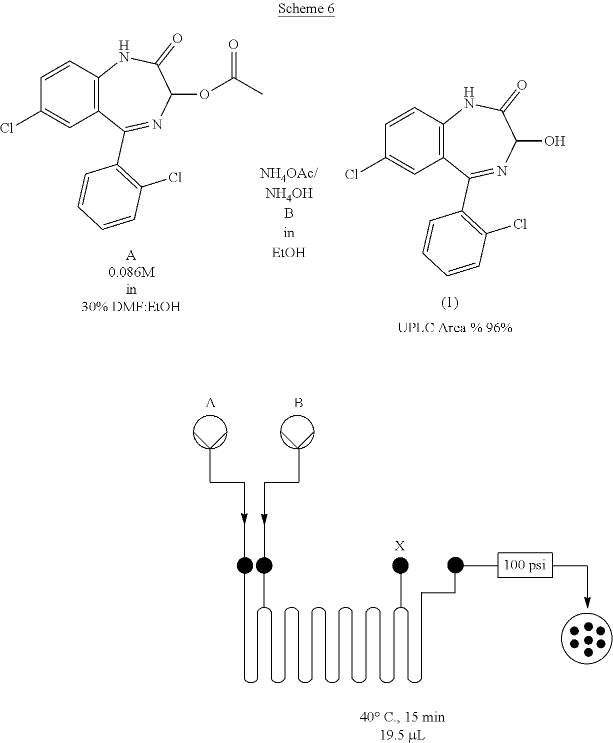

Various mixed solvent systems were tried for the hydrolysis reaction to avoid byproduct formation that occurred in batch experiments. The solvent systems tried were N,N′dimethyl formamide (DMF) and ethanol, DMSO, 2-MeTHF and ACN, DMF:H₂O and methanol and ethanol. The reaction was slow with 2-MeTHF and ACN, whereas byproducts were formed with DMF:H₂O and DMF:EtOH. Hence, the reaction was tried with a mixture of bases such as ammonium hydroxide and ammonium acetate. The optimal condition for flow hydrolysis was achieved using a combination of solvents such as 30% DMF:EtOH and the mixture of bases ammonium hydroxide (65 equiv.), and ammonium acetate (3 equiv.) at a residence time of about 15 min. and at a temperature of about 40° C. to yield lorazepam with 96% purity by UPLC and a yield of 84% without the formation of any byproducts. The presence of ammonium acetate in ammonium hydroxide enhances the rate of lorazepam acetate hydrolysis via the common ion effect or modifying the reaction pH to maintain a clean reaction profile without the formation of detectable byproducts.

The bases that can be used for the hydrolysis reaction can include, but are not limited to, ammonium hydroxide, sodium hydroxide, aluminum hydroxide, magnesium hydroxide, potassium hydroxide, ammonia, 7 M ammonia in MeOH, triazabicyclodecene, triethylamine, and ammonium acetate. The bases can be used in a combination with additives, such as ammonium acetate, sodium acetate, potassium acetate, aluminum acetate dibasic, and acetate buffer.

Examples of the solvents used for the hydrolysis reaction can include, but are not limited to, DMF, N,N′dimethyl acetamide, ethanol, methanol, butanol, iso-propanol, ACN, N-methyl pyrrolidone, water, and a combination thereof. Examples of a combination of solvents can include, but are not limited to, DMF and ethanol, ACN and ethanol, ethanol and water, and N-methyl pyrrolidone and ethanol.

The residence times for the hydrolysis reaction can range from about 1 min. to about 120 min., such as from about 1 min. to 120 min. or 1 min. to about 120 min.

The reaction temperature for the hydrolysis reaction can range from about −20° C. to about 100° C., such as from about −20° C. to 100° C. or −20° C. to about 100° C.

Any suitable continuous flow reactor as is known in the art can be used for the continuous flow synthesis. Examples of the suitable continuous flow reactor includes plug flow reactors, segmented flow reactors, and continuous stirred tank reactors. These reactors can be a glass reactor, a ceramic reactor, a coiled flow inverter reactor, a coiled tubing reactor, a packed bed reactor, an oscillatory, a spinning disc, or a 3D-printed type reactor.

The continuous flow reactor system comprises of one or more mixers, staggered oriented ridge reactor chips, one or more syringe pumps to feed the solutions of reactants and reagents into the reactor, and a software to program recipes for reaction conditions, such as flow rates and temperature conditions. The staggered oriented ridge reactor chips can be chip 3227 19.5 μL or chip 3225 10 μL. The software to program recipes for reaction conditions can be ChemTrix GUI. The software sets the flow rates of the reactions and calculates the residence time based on the volume of the reactor and volumetric flow rates.

Examples of a mixer for reagent mixing for each continuous flow reaction step include, but are not limited to, T-mixers, Y-mixers, static mixers, ultrasonic mixers, staggered oriented ridge mixers, zig-zag mixers, packed bed mixers, and coiled flow inverter mixers.

Using the continuous flow process, the impurities have been reduced for each step in the synthesis with the incorporation of greener solvents and milder reagents. The time scale from batch to flow synthesis has been reduced from days to minutes, and there is an increment in the yield for 4 out of 5 steps compared to batch synthesis.

Provided is a method for a telescoped continuous flow synthesis of delorazepam (4) including performing step (a) and step (b) together in one flow. The method comprises: mixing a solution comprising 2-amino-2′,5-dichlorobenzophenone (2) with a solution comprising an acid scavenger, a solution comprising an acylating agent, and a solution comprising an ammonium reagent in the continuous flow reactor.

Examples of the solvents that can be used in telescoped continuous flow synthesis of delorazepam include, but are not limited to, acetonitrile, 2-MeTHF, ethyl acetate, toluene, N-methyl pyrrolidone, water, dichloromethane, acetone, methanol, ethanol, butanone, dimethyl sulfoxide, acetic acid, and a combination thereof. Examples of the combination of solvents can include, but are not limited to, acetonitrile and water; acetonitrile and N-methyl pyrrolidone; N-methyl pyrrolidone, acetonitrile, and water; acetonitrile, acetic acid, and 2-MeTHF; 2-MeTHF, acetonitrile, and water; and ethanol, acetonitrile, and water. In exemplary embodiments, the most suitable solvent can be acetonitrile and a combination of acetonitrile and water.

The residence time of telescoped continuous flow synthesis of delorazepam can be about 1 min. to about 3 hours, preferably about 10 min. The reaction can be performed in different reactors. The telescoped synthesis optimization was performed using Chemtrix glass reactor chips 3225 (10 μL) and 3227 (19.5 μL) and Coiled Tubing reactor (0.762 mm ID).

Scheme 7 shows the telescoped continuous flow synthesis of Delorazepam (4) in the coiled tubing continuous flow reactor.

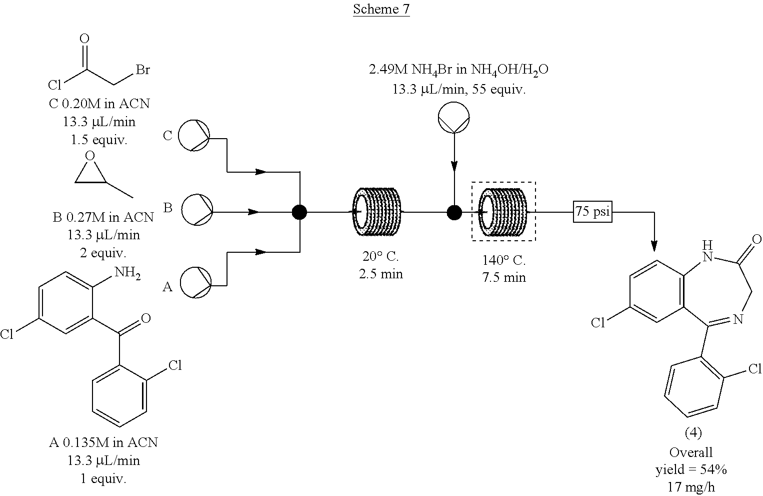

The results from the reactions from all three reactors were summarized in Table 4 below.

TABLE 4

| | | | | UPLC Area % of |
|---|---|---|---|---|
| | Temp. (° C.) | Reactor type for | Reactor type for | Delorazepam 4/ |
| Entry | T₁ + T₂ | Step 1 | Step 2 | Intermediate 9 |
| 1 | 20 + 140 | glass chip reactor | glass chip | clog, red ppt. |
| 2 | 0 + 140 | glass chip reactor | glass chip | Clog |
| 3 | 20 + 140 | glass chip reactor | glass chip in | clog, crystallization of |
| | | | sonication | 6 |
| 4 | 20 + 140 | glass chip reactor | tubing (0.25 mm ID) | clog, red ppt. |
| 5 | 20 + 140 | tubing (0.762 mm | tubing (0.762 mm ID), | Pump stalled |
| | | ID) | T-mixer held at 20° C. | |
| 6 | 20 + 140 | tubing (0.762 mm | tubing (0.762 mm ID), | 90%/10%^{a} |
| | | ID) | T-mixer held at | |
| | | | 140° C. | |
| 7 | 20 + 115 | tubing (0.762 mm | tubing (0.762 mm ID), | 75%/14%^{a} |
| | | ID) | T-mixer held at | |
| | | | 140° C. | |

In a Chemtrix glass reactor, a clog was observed in the second step, where the ammonium bromide/ammonium hydroxide was mixed with the output from step (a) to initiate the cyclization reaction (Table 4, Entries 1-2). This problem was compounded upon placing the second reactor in an ultrasonication bath, leading to the crystallization of halo intermediate (3) inside the staggered oriented ridge mixer of the reactor chip (Table 4, Entry 3). The T-mixer with a PFA reactor tubing (internal diameter 0.25 mm) was employed for the step (b) while keeping step (a) in the glass reactor chip (Table 4, Entry 5), although reactor clogging was still observed. The clog in each of the two cases was removable by flushing the system with 0.2 M HCl solution. Both the steps (a) and (b) were then moved to PFA tubing (internal diameter 0.726 mm). When the outlet of the T-mixer was held at 20° C., the syringe pumps stalled and resulted in a clog. Subsequent experimentation revealed that it was critical to hold the super flangeless nut at the T-mixer outlet at a higher temperature (110° C. or 140° C.) to achieve a consistent flow without any clogging (Table 4, Entry 5-6). An additional impurity was observed during the telescoped synthesis, occurring with a retention time of 5.6 min and an m/z of 305. Delorazepam (4) was isolated from the impurity by extracting it in 3 M HCl, neutralizing it with ammonium hydroxide, and then back extracting with EtOAc to give 4 in 90% purity. Thus, the effective results were obtained in a coiled tubing reactor at higher temperatures.

Scheme 8 illustrates the comparison of batch and continuous flow synthesis of lorazepam (1)

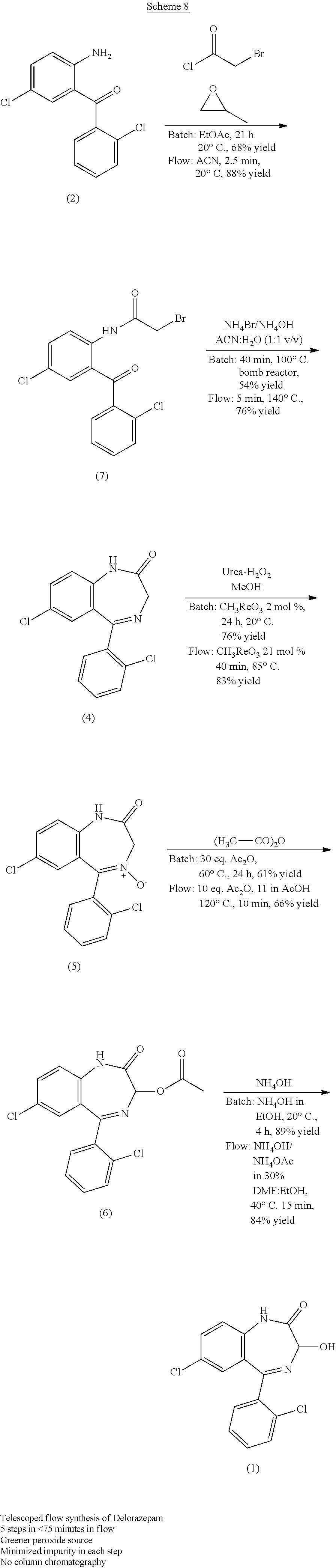

The five-step synthesis of lorazepam (1) was performed in less than 75 minutes of mean residence time summed for each individual step by continuous flow synthesis. Thus, the advantages of continuous flow synthesis are significantly reduced total synthesis time, improved yield of product at each step, minimized impurities, avoided purification methods such as column chromatography, and use of milder, safer, and sustainable reagents and solvents in each step.

With the various embodiments that have been described, it will be apparent to those of ordinary skill in the art that many more embodiments and implementations are possible. Accordingly, the embodiments described herein are examples, not the only possible embodiments and implementations.

### EXPERIMENTAL

The following examples serve to illustrate the present disclosure. The examples are not intended to limit the scope of the claimed invention in any way.

### General Information

All reagents were purchased either from Sigma Aldrich or TCI America and used without further purification. Samples for ¹H-NMR and ¹³C-NMR were analyzed by a Bruker AV-III-500-HD NMR spectrometer. ESI-MS and ESI-MS/MS experiments were performed using a Thermo Fisher TSQ Quantum Access MAX mass spectrometer. For the analysis and processing of data, Thermo Fisher Xcalibur software was utilized. Analytical thin layer chromatography (TLC) was performed on 0.2 mm coated silica gel plates (Thermo Fisher Scientific, MilliporeSigma™ TLC Silica Gel 60 F254). Visualization was accomplished using 254 nm and 365 nm UV light. Column chromatography was carried out using a Biotage SP4 system equipped with normal phase silica Redisep columns (average particle size 35-70 μm, average pore size 60 Å). Ultra-High Pressure Liquid Chromatography-Mass Spectrometry (UPLC) was performed using a Waters Acquity H-Class Plus System. A CORTECS C18 column (2.1 mm×100 mm, pore size 1.6 μm) and a CORTECS VanGuard pre-column (2.1×5 mm×1.6 μm) were installed preceding the analytical C18 column before purging with 90:10 Water:ACN.

### Continuous Flow Chemtrix S1 Reactor

All continuous flow microfluidic experiments were carried out using a Chemtrix Labtrix S1 (Chemtrix, Ltd., Netherlands) system with glass reactor chips 3227 (19.5 μL) or 3225 (10 μL) with staggered oriented ridge mixers. This system is configured with five syringe pumps feeding a microreactor that is positioned onto a Peltier temperature control stage. FEP tubing (0.8 OD×0.25 mm ID, Dolomite Microfluidics) with 1 mL gastight glass syringes (Hamilton, Nevada) were used. The recipes for the reaction conditions are entered into the ChemTrix GUI software. For the telescoped flow synthesis of Delorazepam, PFA tubing ( 1/16″ OD×0.03″ ID, IDEX Health and Science) was used. All the microfluidic parts, including unions, super-flangeless nuts, back-pressure regulators, tubing-sleeves, and T-mixers were purchased from IDEX Health and Science.

### Electrochemical Reactor

Electrochemical experiments were performed using an IKA Electrasyn 2.0 Pro package. Vials (5 mL) with respective electrodes were placed in the vial for the small-scale reaction screening. The experiments were done by applying constant current.

### Batch Synthesis of 2-Bromo-N-(4-chloro-2-(2-chlorobenzoyl)phenyl)acetamide (7)

In a round bottom flask, a solution of 2-amino-2′,5-dichlorobenzophenone (50 mL, 25 mmol, 6.653 g, 1 equiv.) in ethyl acetate was cooled to 0° C. Propylene oxide (3.49 mL, 50 mmol, 2 equiv.) was added and stirred for 2 minutes. Bromoacetyl chloride (2.2 mL, 26.5 mmol, 1.06 equiv.) was added to the solution dropwise using a dropping funnel. The solution was stirred for 21 h at 20° C. The formation of a white precipitate was observed over time. An EtOAc:hexane mixture (2:8 v/v, 10 mL) was added to the solution, resulting in a cloudy solution. The white precipitate was filtered, and the filtrate (white precipitate) was washed with 2% EtOAc: hexane solution to give 6.5 g of the product.

TLC: R_{f}=0.53 in 3% IPA:DCM, Yield: 68%.

¹H NMR (500 MHz, CDCl₃) δ 12.01 (s, 1H), 8.74 (d, J=9.1 Hz, 1H), 7.56 (dd, J=9.1, 2.6 Hz, 1H), 7.49 (d, J=3.5 Hz, 2H), 7.45-7.38 (m, 1H), 7.38-7.33 (m, 2H), 4.06 (s, 2H). ¹³C NMR (126 MHz, CDCl₃) δ 198.45, 166.81, 138.56, 137.76, 135.44, 133.48, 131.88, 131.10, 130.38, 128.92, 128.46, 127.03, 122.89, 122.34, 28.57.

### Continuous Flow Synthesis of 2-Bromo-N-(4-chloro-2-(2-chlorobenzoyl)phenyl)acetamide (7)

Solutions of 2-amino-2′,5-dichlorobenzophenone (200 mg, 0.751 mmol, 1 equiv., 0.135M) in ACN (5.6 mL), propylene oxide (241 μL, 0.27M, 2 equiv.) in ACN (12.75 mL), and bromoacetyl chloride (125 μL, 0.169M, 1.5 equiv.) in ACN (7.52 mL) were prepared. Reactor chip 3225 (10 μL with a staggered oriented ridge mixer) was placed in the reactor holder along with the inlet and outlet lines and then mounted on the Peltier stage. Each of the solutions were loaded into 1 mL Hamilton syringes and mounted onto syringe pumps 1, 2, and 3, respectively. Outlet 4 was blocked by a blind plug, and the outlet of the reactor was channeled into the carousel unit. The recipe for the reaction was input using the Chemtrix software where flow rates for the benzophenone and propylene oxide were set to 2 μL/min each and R_{T}=2.5 minute. Waste was collected until 5× residence time volumes had passed before initiating sample collection. The vial in the carousel contained 100 μL 0.25 M Na₂CO₃ and 900 μL ACN prior to product collection; 50 μL of the product solution was collected in the carousel vials. For UPLC and UPLC-MS analysis, the solutions were diluted 1:10 in ACN, followed by filtration via 0.2 μm PTFE syringe filters. TLC: R_{f}=0.53 in 3% IPA:DCM, Yield=88%. (Calibration curves were developed to determine the yield).

### Batch Synthesis of 7-Chloro-5-(2-chlorophenyl)-1,3-dihydro-2H-benzo[e][1,4]diazepin-2-one (4)

In a bomb reactor, 140 mg (0.36 mmol, 1 equiv.) of 2-bromo-N-(4-chloro-2-(2-chlorobenzoyl)phenyl)acetamide (7) was added to 3 mL ACN along with a stir bar. A separate solution of 2360 mg NH₄Br (24 mmol, 67 equiv.) dissolved in 2.8 mL of 30% NH₄OH in 2.1 mL DI H₂O was prepared and then added to the ACN solution. The reaction mixture was stirred for 40 minutes in an oil bath at 100° C. The reaction mixture was then extracted with 15 mL EtOAc and washed with 4 mL DI H₂O (3×). The organic layer was dried over Na₂SO₄, filtered, and concentrated in vacuo to give 59 mg Delorazepam.

TLC: R_{f}=0.2 in 3% IPA:DCM, Yield: 54%.

¹H NMR (500 MHz, CDCl₃) δ 9.75 (s, 1H), 7.54-7.47 (m, 1H), 7.40 (dd, J=11.3, 8.9 Hz, 4H), 7.13 (d, J=8.7 Hz, 1H), 7.04 (d, J=2.5 Hz, 1H), 4.38 (s, 2H).

¹³C NMR (126 MHz, CDCl₃) δ 171.43, 169.46, 138.33, 136.64, 133.22, 132.01, 131.07, 131.04, 130.22, 129.25, 129.21, 129.17, 127.05, 122.65, 53.40.

### Continuous Flow Synthesis of 7-Chloro-5-(2-chlorophenyl)-1,3-dihydro-2H-benzo[e][1,4]diazepin-2-one (4)

A solution of 2-bromo-N-(4-chloro-2-(2-chlorobenzoyl)phenyl)acetamide (7) (70 mg, 0.180 mmol, 1 equiv.) in ACN (4 mL, 0.045M) was prepared. A solution of 1.16 g NH₄Br (12.2 mmol, 2.49M, 67.7 equiv.) in 30% NH₄OH (1.4 mL) and DI H₂O (3.5 mL) was prepared. Each of the solutions was loaded into 1 mL Hamilton syringes and mounted onto syringe pumps 1 and 2, respectively, of the S1 system. Reactor chip 3227 (19.5 μL with a staggered oriented ridge mixer) was placed in the reactor holder along with the inlet and outlet lines and then mounted onto the Peltier stage. Outlet 4 was blocked by a blind plug and the outlet of the reactor was channeled into the carousel unit. The recipe for the reaction was input using the Chemtrix software. The flow rates for each syringe were set to 1.3 μL/min, R_{T}=7.5 min, and a temperature of 140° C. Waste was collected until 5× residence time volumes had passed before initiating sample collection. The vial in the carousel contained 900 μL ACN prior to product collection; 100 μL of the product solution was collected in the carousel vials. For UPLC and UPLC-MS analysis, the solutions were diluted 1:10 in ACN, followed by filtration via 0.2 μm PTFE syringe filters. TLC: R_{f}=0.2 in 3% IPA:DCM, Yield: 76%. (Calibration curves were developed to determine the yield).

### Telescoped Continuous Flow Synthesis of Delorazepam 7-Chloro-5-(2-chlorophenyl)-1,3-dihydro-2H-benzo[e][1,4]diazepin-2-one (4)

Solutions of 0.134 M of 2-amino-2′,5-dichlorobenzophenone in ACN, 0.27 M propylene oxide in ACN, and 0.2 M bromoacetyl chloride in ACN, and 2.4 M NH₄Br with 1.4 mL 30% NH₄OH in 3.5 mL DI H₂O were prepared. Each of them was loaded into 2.5 mL Hamilton syringes and mounted onto a Harvard syringe pump. The solutions were then fed into a 4-way mixer via PFA tubing (0.762 mm internal diameter) at 20° C. and R_{T}=2.5 minutes to initiate the first N-acylation step. The outlet line of the 4-way mixer was then fed into a T-mixer that was also combined with the NH₄Br/NH₄OH solution to initiate the second cyclization step. The outlet of the T-mixer was held at 140° C. by placing it in a heated oil bath. The tubing was coiled to give a residence time of 7.5 minutes. A 75 psi back pressure regulator was attached inline at the outlet, just before the product collection port. The flow rates for each syringe were set to 13.3 μL/min, and the reaction solution was delivered to waste for 5× residence times (˜50 minutes) prior to initiation of sample collection. A total of 137 min of collection was timed, after which the output was transferred back to waste followed by flushing the lines with ACN. The reaction solution was syringe filtered (0.2 μm PTFE) prior to analysis by UPLC. The reaction solution was worked up by extracting the 2 mL reaction mixture in 10 mL 3 M HCl and 3 mL EtOAc and then back extracting the aqueous layer in EtOAc after neutralizing with 0.5 mL 30% NH₄OH. A total of 41 mg of the final product was obtained.

TLC: R_{f}=0.1 in 1:1 EtOAc:Hexane, Overall Isolated Yield: 54%.

¹H NMR (500 MHz, CDCl₃) δ 9.75 (s, 1H), 7.54-7.47 (m, 1H), 7.40 (dd, J=11.3, 8.9 Hz, 4H), 7.13 (d, J=8.7 Hz, 1H), 7.04 (d, J=2.5 Hz, 1H), 4.38 (s, 2H).

¹³C NMR (126 MHz, CDCl₃) δ 171.43, 169.46, 138.33, 136.64, 133.22, 132.01, 131.07, 131.04, 130.22, 129.25, 129.21, 129.17, 127.05, 122.65, 53.40. information for the image).

### Batch Synthesis of 7-Chloro-5-(2-chlorophenyl)-2-oxo-2,3-dihydro-1H-benzo[e][1,4]diazepine 4-oxide (5)

In a round bottom flask, 500 mg (1.64 mmol, 1 equiv.) of delorazepam was dissolved in 16 mL anhydrous MeOH. The solution was stirred vigorously using a vortex mixer until it was completely dissolved. Urea-hydrogen peroxide (462.8 mg, 4.92 mmol, 3 equiv.) was then added, followed by 8.2 mg (0.033 mmol, 0.02 equiv.) of CH₃ReO₃. The reaction mixture was stirred for −21 hours until the disappearance of the starting material was observed by TLC. The MeOH was removed by rotary evaporation, and the reaction mixture was extracted with EtOAc:H₂O. The organic layer was washed with DI H₂O (3×), dried with anhydrous Na₂SO₄, filtered, and concentrated in vacuo to give 400 mg of product as a white solid.

TLC: R_{f}=0.1 in 50:50 EtOAc:Hexane, Yield: 76%.

¹H NMR (500 MHz, DMSO) δ 11.26 (s, 1H), 7.57 (d, J=6.8 Hz, 1H), 7.53-7.42 (m, 4H), 7.30 (d, J=8.9 Hz, 1H), 6.77 (d, J=2.5 Hz, 1H), 4.80-4.54 (m, 2H).

¹³C NMR (126 MHz, DMSO) δ 165.35, 138.91, 136.09, 133.47, 133.17, 132.28, 131.74, 130.36, 130.24, 128.44, 128.09, 126.05, 124.18, 68.24.

### Continuous Flow Synthesis of 7-Chloro-5-(2-chlorophenyl)-2-oxo-2,3-dihydro-1H-benzo[e][1,4]diazepine 4-oxide (5)

All the solid compounds were flushed with argon followed by vacuum and backfilled with argon three times prior to the preparation of the solutions. Solutions of delorazepam (4, 61 mg, 0.2 mmol, 0.1M) in 2 mL anhydrous MeOH, Urea-H₂O₂ (112.8 mg, 1.2 mmol, 0.6M) in 2 mL anhydrous MeOH and CH₃ReO₃ (21 mg, 0.064 mmol, 0.032M) in 2 mL anhydrous MeOH were prepared. Reactor chip 3227 (19.5 μL with a staggered oriented ridge) was mounted on the Chemtrix Peltier stage along with the inlet and outlet lines. Each of the solutions were loaded into 1 mL Hamilton syringes and then mounted onto syringe pumps 1, 2, and 3, respectively. The lines were fed into the reactor chip via a T-mixer joining the Urea-H₂O₂ and CH₃ReO₃ lines. A 500 psi ultra-low volume back pressure regulator was placed at the end of the line and channeled into the carousel unit for sample collection. The recipe for the reaction was input using the Chemtrix software with the temperature set to 85° C. and R_{T}=40 minutes. Flow rate for syringes 1, 2, and 3 were set to 0.243 μL/min, 0.122 μL/min, and 0.122 μL/min, respectively. Anhydrous MeOH was placed in the quench line at a flow rate of 0.243 L/min. Waste was collected until 5× residence times prior to sample collection. The vials in the carousel were pre-loaded with 100 μL sat. NaHSO₃ solution and 500 μL ACN prior to collection of 100 μL product solution in the 1.5 mL vials. The UPLC and UPLC-MS analysis was performed after a 1:10 dilution in ACN and filtration of the solution via 0.2 μm PTFE syringe filter. TLC: R_{f}=0.1 in 50:50 EtOAc:Hexane, Yield: 83% (Calibration curves were developed to determine the yield).

### Batch Synthesis of 7-Chloro-5-(2-chlorophenyl)-2-oxo-2,3-dihydro-1H-benzo[e][1,4]diazepin-3-yl acetate (Compound 6)

In a round bottom flask, 597 mg (1.86 mmol, 1 equiv.) of Delorazepam N-oxide 5 was added. Acetic anhydride (5.57 mL, 58.93 mmol, 31.7 equiv.) was added, and the solution was stirred at 60° C. for 24 hours. The reaction was brought to 20° C. before the addition of 3.35 mL water. The reaction solution was filtered to give 430 mg of the product as a white solid.

TLC: R_{f}=0.36 (50:50 EtOAc:Hexane), Yield=63%.

¹H NMR (500 MHz, DMSO) δ 11.24 (s, 1H), 7.68-7.58 (m, 2H), 7.56-7.47 (m, 3H), 7.30 (d, J=8.8 Hz, 1H), 6.96 (d, J=2.5 Hz, 1H), 5.82 (s, 1H), 2.19 (s, 3H).

¹³C NMR (126 MHz, DMSO) δ 170.03, 165.09, 164.76, 137.65, 137.21, 132.93, 132.30, 132.22, 131.98, 130.32, 129.01, 128.49, 128.01, 127.89, 123.92, 85.83, 21.20.

### Continuous Flow Synthesis of 7-Chloro-5-(2-chlorophenyl)-2-oxo-2,3-dihydro-1H-benzo[e][1,4]diazepin-3-yl acetate (6)

Solution of 7-chloro-5-(2-chlorophenyl)-2-oxo-2,3-dihydro-1H-benzo[e][1,4]diazepine 4-oxide (5) (44.8 mg, 0.139 mmol, 1 equiv.) in acetic acid (0.7 mL, 0.198M) was prepared. The second solution was neat acetic anhydride. A glass reactor chip 3227 (19.5 μL with a staggered oriented ridge) was placed on the Chemtrix Peltier stage along with the inlet and outlet lines. Each of the solutions were loaded into 1 mL Hamilton syringes and mounted onto syringe pumps 1 and 2, respectively. A 500 psi ultra-low volume back pressure regulator was placed at the end of the line and channeled into the carousel unit for sample collection. The recipe for the reaction was input using the Chemtrix software where the residence time and temperature were set to 10.14 min and 120° C., respectively. The flow rate of pump 1 was set to 0.47 μL/min, and the flow rate of pump 2 was set to 1.453 μL/min. Waste was collected for 5× residence times prior to sample collection. The vials in the carousel were pre-loaded with 900 μL ACN prior to product collection and 100 μL solution was collected. Each sample was frozen at −80° C. after collection. The UPLC and UPLC-MS analysis was performed after 1:10 dilution in ACN and filtration of the solution via 0.2 μm PTFE syringe filter. TLC: R_{f}=0.36 (50:50 EtOAc:Hexane), Yield: 66% (calibration curves were developed to determine the yield).

### Batch Synthesis of Lorazepam (1)

In a round bottom flask, 153 mg (0.42 mmol, 1 equiv.) Lorazepam acetate was stirred in 4.3 mL ethanol. NH₄OH (0.5 mL, 30%) was added dropwise at 20° C. over a period of 5 minutes. After 1 h, an additional 0.3 mL 30% NH₄OH was added. The solution was stirred at 20° C. for a total of 4 hours while monitoring for the disappearance of starting material. The reaction solution was then extracted in 30 mL DCM and washed with 9 mL DI H₂O (3×). The organic layer was concentrated in vacuo. The product was dried under high vacuum at 0° C. for 2 h prior to NMR analysis.

TLC: R_{f}=0.16 (50:50 EtOAc:Hexane), Yield: 89%.

¹H NMR (500 MHz, DMSO) δ 10.94 (s, 1H), 7.60 (dd, J=8.7, 2.5 Hz, 2H), 7.57-7.45 (m, 3H), 7.25 (d, J=8.7 Hz, 1H), 6.94 (d, J=2.4 Hz, 1H), 6.43 (d, J=8.9 Hz, 1H), 4.84 (d, J=8.4 Hz, 1H).

¹³C NMR (126 MHz, DMSO) δ 169.11, 161.98, 137.77, 137.12, 131.82, 131.79, 131.39, 131.30, 129.74, 128.70, 127.68, 127.43, 126.84, 123.11, 82.86.

### Continuous Flow Synthesis of Lorazepam (1)

A solution of Lorazepam acetate (6) (30 mg, 0.083 mmol, 1 equiv.) in DMF:EtOH (0.955 mL, 0.0865M, 30% v/v) was prepared. A second solution of NH₄OH (0.4 mL, 65 equiv., 30%) and NH₄OAc (20 mg, 0.259 mmol, 3.12 equiv.) in EtOH (0.65 mL) was prepared to give a final volume of 1.05 mL. A glass reactor chip 3227 (19.5 μL with a staggered oriented ridge) was placed on the Chemtrix Peltier stage along with the inlet and outlet lines. Each of the solutions were loaded into 1 mL Hamilton syringes and then mounted onto syringe pumps 1 and 2, respectively. A 100 psi back pressure regulator was placed at the end of the line and channeled into the carousel unit for sample collection. The recipe for the reaction was input using the Chemtrix software, where the residence time and temperature were set to 15 minutes and 40° C., respectively. The flow rates of pump 1 and pump 2 were set to 0.65 μL/min. Waste was collected for 5× residence times prior to sample collection. The vials in the carousel were pre-loaded with 500 μL ACN prior to product collection and 70 μL solution was collected. The UPLC and UPLC-MS analysis was performed after filtration of the solutions via 0.2 μm PTFE syringe filters. TLC: R_{f}=0.16 (50:50 EtOAc:Hexane), Yield: 84% (Calibration curves were developed to determine the yield).

Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

Those skilled in the art will recognize that numerous modifications can be made to the specific implementations described above. The implementations should not be limited to the particular limitations described. Other implementations may be possible.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A method for synthesis of (7-chloro-5-(2-chlorophenyl)-3-hydroxy-1,3-dihydro-2H-benzo[e][1,4]diazepin-2-one) lorazepam (1) 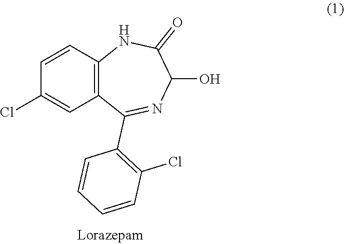 [SMILES: O=C1Nc2ccc(Cl)cc2C(c2ccccc2Cl)=NC1O]
in a continuous flow reactor which method comprises:
(a) acylating 2-amino-2′,5-dichlorobenzophenone (2) by mixing a solution comprising (2) 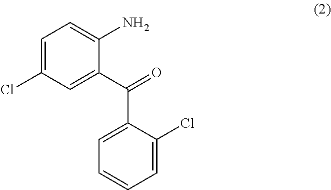 [SMILES: Nc1ccc(Cl)cc1C(=O)c1ccccc1Cl]
with a solution comprising an acylating agent, and a solution comprising an acid scavenger to yield 2-halo-N-(4-chloro-2-(2-chlorobenzoyl)phenyl)acetamide (halo intermediate, (3)); 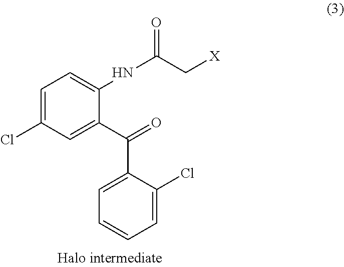
wherein X is a halogen comprising Cl or Br;
(b) cyclizing the halo intermediate (3) by mixing a solution comprising the halo intermediate (3) with a solution comprising an ammonium reagent to yield 7-chloro-5-(2-chlorophenyl)-1,3-dihydro-2H-benzo[e][1,4]diazepin-2-one (delorazepam, (4)); 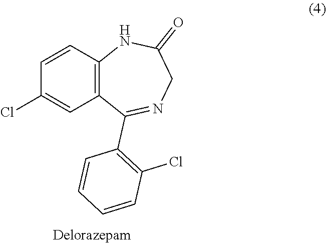 [SMILES: O=C1CN=C(c2ccccc2Cl)c2cc(Cl)ccc2N1]
(c) oxidizing delorazepam (4) by mixing a solution comprising delorazepam (4) with a solution comprising a peroxide reagent and a solution comprising a rhenium oxide catalyst to yield 7-chloro-5-(2-chlorophenyl)-2-oxo-2,3-dihydro-1H-benzo[e][1,4]diazepine 4-oxide (delorazepam N-oxide, (5)); 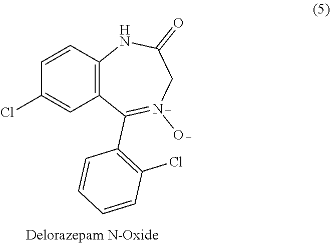
(d) mixing a solution comprising delorazepam N-oxide (5) with a solution comprising an acylating reagent for performing Polonovski-type rearrangement to yield 7-chloro-5-(2-chlorophenyl)-2-oxo-2,3-dihydro-1H-benzo[e][1,4]diazepin-3-yl acetate (lorazepam acetate, (6)); 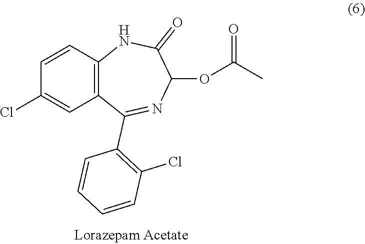 [SMILES: CC(=O)OC1N=C(c2ccccc2Cl)c2cc(Cl)ccc2NC1=O]
and
(e) hydrolyzing lorazepam acetate (6) by mixing a solution comprising lorazepam acetate (6) and a solution comprising a base and an additive to yield lorazepam (1).

2. The method of claim 1, wherein the continuous flow reactor is selected from a plug flow reactor, a segmented flow reactor and a continuous stirred tank reactor and/or the reactor can be a glass reactor, a ceramic reactor, a coiled flow inverter reactor, a coiled tubing reactor, a packed bed reactor, an oscillatory reactor, a spinning disc, or a 3D-printed type reactor.

3. (canceled)

4. The method of claim 2, wherein the continuous flow reactor further comprises a mixer selected from a T-mixer, a Y-mixer, a static mixer, an ultrasonic mixer, a staggered oriented ridge mixer, a zig-zag mixer, a packed bed mixer, and a coiled flow inverter mixer.

5. The method of claim 1, wherein the acylating reagent used in step (a) is selected from bromoacetyl bromide, bromoacetyl chloride, 2-bromoacetic acid, bromoacetic anhydride, chloroacetyl chloride, and ethyl bromoacetate and/or the acid scavenger in step (a) is selected from propylene oxide, ethylene oxide, and butylene oxide.

6. (canceled)

7. The method of claim 1, wherein the ammonium reagent in step (b) is selected from ammonium chloride, ammonium iodide, ammonium bromide, ammonium hydroxide, ammonium acetate, and a combination of two or more ammonium reagents.

8. The method of claim 1, wherein the peroxide reagent in step (c) is selected from urea-hydrogen peroxide, magnesium monoperoxyphthalate, H₂O₂, peroxyacetic acid, tert-butyl hydroperoxide, and sodium percarbonate and/or the rhenium oxide catalyst in step (c) is selected from CH₃ReO₃ and Re₂O₇.

9. (canceled)

10. The method of claim 1, wherein the acylating reagent in step (d) is selected from acetic anhydride, trifluoroacetic anhydride, and acetyl chloride.

11. The method of claim 1, wherein the base in step (e) is selected from ammonium hydroxide, sodium hydroxide, potassium hydroxide, and ammonia and/or the additive in step (e) is selected from ammonium acetate, sodium acetate, potassium acetate, and aluminum acetate dibasic.

12. (canceled)

13. The method of claim 1, wherein the solutions in step (a) are prepared in a solvent selected from acetonitrile, 2-MeTHF, ethyl acetate, toluene, N-methyl pyrrolidone, water, dichloromethane, acetone, and a combination of two or more solvents.

14. The method of claim 1, wherein the solutions in step (b) are prepared in a solvent selected from acetonitrile, N-methyl pyrrolidone, methanol, ethyl acetate, acetone, ethanol, toluene, dicholormethane, butanone, water, dimethyl sulfoxide, acetic acid, N-methyl pyrrolidone, 2-MeTHF, and a combination of two or more solvents.

15. The method of claim 1, wherein the solutions in step (c) are prepared in a solvent selected from anhydrous methanol, methanol, 2-MeTHF, ethyl acetate, ethanol, propanol, iso-propanol, butanol, acetone, acetic acid, and a combination of two or more solvents.

16. The method of claim 1, wherein the solutions in step (d) are prepared in a solvent selected from acetic acid, ethyl acetate, acetonitrile, toluene, dichloromethane, water, methanol, ethanol, toluene, dichloromethane, and a combination of two or more solvents.

17. The method of claim 1, wherein the solutions in step (e) are prepared in a solvent selected from N,N′-dimethyl formamide (DMF), N,N′-dimethyl acetamide, ethanol, methanol, butanol, iso-propanol, water, acetonitrile, N-methyl pyrrolidone, and a combination of two or more solvents.

18. The method of claim 1, wherein the step (a) is carried out in a residence time range of about 30 seconds to about 30 minutes.

19. The method of claim 1, wherein the step (a) is carried out at in a temperature range from about −20° C. to about 100° C.

20. The method of claim 1, wherein the step (b) is carried out in a residence time range of about 30 seconds to about 60 minutes and/or in a temperature range from about 20° C. to about 180° C.

21. (canceled)

22. The method of claim 1, wherein the step (c) is carried out in a residence time range from about 5 minutes to 3 hours and/or in a temperature range from about 20° C. to about 150° C.

23. (canceled)

24. The method of claim 1, wherein the Polonovski-type rearrangement is carried out in a residence time range from about 1 minute to about 30 minutes and/or in a temperature range from about 50° C. to about 150° C.

25. (canceled)

26. The method of claim 1, wherein hydrolysis of lorazepam acetate is carried out in a residence time range from about 1 minute to about 120 minutes and/or in a temperature range from about −20° C. to about 100° C.

27. (canceled)

28. A reagent for N-oxidation of 7-chloro-5-(2-chlorophenyl)-1,3-dihydro-2H-benzo[e][1,4]diazepin-2-one (delorazepam (4)) to yield delorazepam N-oxide (5) comprising a combination of a peroxide reagent and a rhenium oxide catalyst.

29-30. (canceled)

31. An ammonium reagent for cyclization of a 2-halo-N-(4-chloro-2-(2-chlorobenzoyl)phenyl)acetamide (halo intermediate (3)) to yield 7-chloro-5-(2-chlorophenyl)-1,3-dihydro-2H-benzo[e][1,4]diazepin-2-one (delorazepam, (4)) comprising a mixture of ammonium bromide and ammonium hydroxide, or a mixture of ammonium iodide, ammonium acetate, and ammonium chloride.
